(19) United States
(12) Patent Application Publication
(10) Pub. No.: US 2024/0315287 A1
(43) Pub. Date: Sep. 26, 2024

(54) COMPOSITION AND COMPOSITE ARTICLE FOR FORMING NITRIC OXIDE

(71) Applicant: Sterile State, LLC, Farmington Hills, MI (US)

(72) Inventor: Megan Cecelia Frost, Farmington Hills, MI (US)

(73) Assignee: Sterile State, LLC, Farmington Hills, MI (US)

(21) Appl. No.: 18/693,129

(22) PCT Filed: Oct. 20, 2022
(86) PCT No.: PCT/US22/47308

§ 371 Date: Mar. 18, 2024

Related U.S. Application Data
(60) Provisional application No. 63/271,103, filed on Oct. 22, 2021.

(51) Int. Cl. A23L 3/358; A01N 59/00

(52) U.S. Cl. CPC A23L 3/358; A01N 59/00

(57) ABSTRACT

A composite article is provided herein. The composite article includes a source layer and an activation layer overlying the source layer. The source layer includes a nitric oxide precursor and the activation layer includes a thiol-containing compound. The nitric oxide precursor and the thiol-containing compound are capable of reacting in the presence of a solvent to form nitric oxide.

### CROSS-REFERENCE TO RELATED APPLICATIONS

This application is an International Application which claims priority to Provisional Patent Application No. 63/271,103, filed on Oct. 22, 2021, the entire content of which is incorporated by reference in its entirety.

### FIELD OF THE INVENTION

The present disclosure generally relates to a composition and composite article includes a source layer and an activation layer capable of forming nitric oxide for food preservation and sanitation.

### BACKGROUND

A variety of products and articles, including, for example, medical instruments, devices, and equipment, must be sterilized prior to use to prevent bio-contamination of a wound site, a sample, an organism, or the like. A number of sterilization processes are used which involve contacting the product or article with a sterilant. Examples of such sterilants include dinitrogen tetraoxide, nitric oxide, steam, ethylene oxide, hydrogen peroxide, dry heat, and the like. Conventional methods for forming nitric oxide use catalytic and enzymatic generation of nitric oxide from nitrite.

There is a need to create very stable, long-term nitric oxide releasing materials that are temperature insensitive and light insensitive. The temperature insensitivity will allow several manufacturing techniques to be used (such as extrusion, thermal curing, etc. that are not compatible with nearly all currently used nitric oxide generating materials). Additionally, the temperature and light insensitivity opens the potential applications of nitric oxide releasing materials to a wide array of potential applications where nitric oxide release may be desirable to reduce microbial loads the cause odors, spoil food, sustain mold and fungal growth, etc.

Accordingly, there remains an opportunity for improved compositions and composite articles for capable of forming nitric oxide for food preservation and sanitation.

### BRIEF SUMMARY OF THE INVENTION

A composite article is provided herein. The composite article includes a source layer and an activation layer overlying the source layer. The source layer includes a nitric oxide precursor and the activation layer includes a thiol-containing compound. The nitric oxide precursor and the thiol-containing compound are capable of reacting in the presence of a solvent to form nitric oxide.

The inventors contemplate a composite article including a nitric oxide precursor and a thiol-containing compound that will react to form gas phase nitric oxide in a controllable manner to allow tuning of the release of the nitric oxide depending on the specific desired applications. Nitrosothiol (RSNO) is formed in a polymer matrix under very mild conditions and then reduced to form nitric oxide. In particular, the inventors contemplate a composite article or composition that achieves thermally stable, light stable nitric oxide releasing materials. In various embodiments, the composite article or composition includes a carrier, a nitric oxide precursor, a thiol-containing compound, a catalyst, and various functional layer that can be utilized depending on the particular application (e.g., food packaging for meat, chicken, fish, fruits, vegetables, etc.).

From another perspective, a solvent (e.g., is absorbed into the composite article or composition. The solvent solubilizes the nitric oxide precursor (e.g, sodium nitrite). The nitric oxide precursor is then mobilized in the solvent and flows over the thiol-containing compound (e.g, reduced glutathione). The nitric oxide precursor and the thiol-containing compound react to form an unstable S-nitrosothiol (e.g., S-nitrosoglutathione) in situ that then reduces to form nitric oxide. The nitric oxide diffuses out of the composite article to interact with microorganisms in the area around the composite article.

Control over the composite article release characteristics of this material can come from many aspects of this system that will impact the size of the source layer, the speed of solubilizing the source layer, the concentration and identity of the thiol-containing compound, the characteristics of the metering layers, the number and arrangement of layers serving each of these purposes, the solvent uptake of the various layers present, and the presence of additives such as acid, reducing/oxidizing agents, etc.

Furthermore, the presence of the catalyst (e.g., transition metal) impacts the rate of reduction of the RSNO. Moreover, control of the water uptake and thickness of the layers can be used to modulate the nitric oxide release. Other solvents, such as methanol, ethanol, ethyl acetate, acetone, THF, salt water, etc. can also be used in combination with water, or alternative to water, to solubilize the nitric oxide precursor.

In one exemplary embodiment, a carrier (e.g., cellulose or ethylene vinyl acetate) is saturated with an aqueous solution containing sodium nitrite to form the source layer. A second carrier (e.g., cellulose or ethylene vinyl acetate) is saturated with an aqueous solution containing reduced glutathione to form the activation layer. When the layers are stacked on top of each other to form the composite article and the composite article is exposed to water, nitric oxide is generated due to the reduction of S-nitrosoglutathione, which then rapidly decomposes to release the nitric oxide.

In another exemplary embodiment, the activation layer further includes trace zinc chloride. The presence of the transition metal modifies the rate of nitric oxide generation and the release profile. The trace zinc chloride may be combined with the glutathione in activation layer or present in a separate catalyst layer.

In yet another embodiment, sodium nitrite is mixed into a silicone sealant that is in an uncured form that also contains glutathione in the same carrier. The sodium nitrite may be incorporated directly with the sealant, or in vessels, micelles or capsules within the sealant. The catalyst, such as a transition metal (e.g., zinc chloride) or reducing agent (e.g., ascorbic acid), may be combined with the sealant. Once the sealant is applied and cures, water exposure can solubilize the sodium nitrite and thus promote a reaction with the thiol-containing compound to form S-nitrosothiol that reduces to form nitric oxide. The nitric oxide can prevent mildew formation on the sealant.

### BRIEF DESCRIPTION OF THE DRAWINGS

FIGS. 1-9 are diagrams illustrating non-limiting embodiments of a composite article;

FIG. 10 is a chart illustrating nitric oxide formation by a non-limiting embodiment of the composite article of FIGS. 1-9; and

FIG. 11 is another chart illustrating nitric oxide formation by a non-limiting embodiment of the composite article of FIGS. 1-9.

### DETAILED DESCRIPTION

Except in the examples, or where otherwise expressly indicated, all numerical quantities in this description indicating amounts of material or conditions of reaction and/or use are to be understood as modified by the word “about” in describing the broadest scope of the disclosure. In various embodiments, the terms “about” and “approximately”, when referring to a specified, measurable value (such as a parameter, an amount, a temporal duration, and the like), is meant to encompass the specified value and variations of and from the specified value, such as variations of +/−10% or less, alternatively +/−5% or less, alternatively +/−1% or less, alternatively +/−0.1% or less of and from the specified value, insofar as such variations are appropriate to perform in the disclosed embodiments. Thus the value to which the modifier “about” or “approximately” refers is itself also specifically disclosed.

Practice within the numerical limits stated is generally preferred. Also, unless expressly stated to the contrary: percent, “parts of,” and ratio values are by weight; the description of a group or class of materials as suitable or preferred for a given purpose in connection with the invention implies that mixtures of any two or more of the members of the group or class are equally suitable or preferred; description of constituents in chemical terms refers to the constituents at the time of addition to any combination specified in the description, and does not necessarily preclude chemical interactions among the constituents of a mixture once mixed; the first definition of an acronym or other abbreviation applies to all subsequent uses herein of the same abbreviation and applies mutatis mutandis to normal grammatical variations of the initially defined abbreviation; and, unless expressly stated to the contrary, measurement of a property is determined by the same technique as previously or later referenced for the same property.

It must also be noted that, as used in the specification and the appended claims, the singular form “a,” “an,” and “the” comprise plural referents unless the context clearly indicates otherwise. For example, reference to a component in the singular is intended to comprise a plurality of components.

As used herein, an “embodiment” means that a particular feature, structure or characteristic is included in at least one or more manifestations, examples, or implementations of this invention. Furthermore, the particular features, structures or characteristics may be combined in any suitable manner, as would be apparent to a person skilled in the art. Combinations of features of different embodiments are all meant to be within the scope of the invention, without the need for explicitly describing every possible permutation by example. Thus, any of the claimed embodiments can be used in any combination.

As used herein, the term “weight percent” (and thus the associated abbreviation “wt. %”) typically refers to a percent by weight expressed in terms of a weight of dry matter. As such, it is to be appreciated that a wt. % can be calculated on a basis of a total weight of a composition, or calculated from a ratio between two or more components/parts of a mixture (e.g., a total weight of dry matter).

As used herein, the term “substantially” refers to the complete, or nearly complete, extent or degree of an action, characteristic, property, state, structure, item, or result. As an arbitrary example, an object that is “substantially” enclosed would mean that the object is either completely enclosed or nearly completely enclosed so as to have the same overall result as if the object were completely enclosed.

The drawings are semi-diagrammatic and not to scale and, particularly, some of the dimensions are for the clarity of presentation and are shown exaggerated in the drawings. Similarly, although the views in the drawings for ease of description generally show similar orientations, this depiction in the drawings is arbitrary. Generally, composite articles can be operated in any orientation. As used herein, it will be understood that when a first element or layer is referred to as being “over,” “overlying,” “under,” or “underlying” a second element or layer, the first element or layer may be directly on the second element or layer, or intervening elements or layers may be present where a straight line can be drawn through and between features in overlying relationship. When a first element or layer is referred to as being “on” a second element or layer, the first element or layer is directly on and in contact with the second element or layer. Further, spatially relative terms, such as “upper,” “over,” “lower,” “under,” and the like, may be used herein for ease of description to describe one element or feature's relationship to another element(s) or feature(s) as illustrated in the figures. It will be understood that the spatially relative terms are intended to encompass different orientations of the composite article in use or operation in addition to the orientation depicted in the figures. For example, if the composite article in the figures is turned over, elements described as being “under” other elements or features would then be oriented “above” the other elements or features. Thus, the exemplary term “under” can encompass either an orientation of above or below. The composite article may be otherwise oriented (rotated 90 degrees or at other orientations) and the spatially relative descriptors used herein may likewise be interpreted accordingly.

Throughout this disclosure, where publications are referenced, the disclosures of these publications in their entireties are hereby incorporated by reference into this disclosure to more fully describe the state of the art to which this disclosure pertains.

The following detailed description is merely illustrative in nature and is not intended to limit the embodiments of the subject matter or the application and uses of such embodiments. Furthermore, there is no intention to be bound by any expressed or implied theory presented in the preceding technical field, background, brief summary or the following detailed description.

A composite article 10 and a composition are provided herein that are capable of forming nitric oxide. In various embodiments, a mixture of nitric oxide and air will react, resulting in a mixture containing many different oxides of nitrogen. Specifically, the addition of nitric oxide to air, or air to nitric oxide, results in the formation of nitric dioxide when nitric oxide reacts with the oxygen in air. The concentration of each nitrogen-oxide species that is present in a mixture may vary with temperature, pressure, and initial concentration of the nitric oxide.

Nitric oxide is lipid soluble and has the ability to disrupt the lipid membranes of microorganisms. Furthermore nitric oxide may inactivate thioproteins thereby disrupting the functional proteins of microbes. Nitrogen dioxide is more water soluble than nitric oxide. Finally, nitric oxide and nitrogen dioxide are effective disruptors of DNA, causing strand breaks and other damage leading to an inability for the cell to function.

As used herein, the term “nitric oxide” or “NO” means the NO free radical or NOₓ. As used herein, the term NOₓ is an abbreviation for nitrogen oxides or the oxides of nitrogen, which are the oxides formed by nitrogen in which nitrogen exhibits each of its positive oxidation numbers from +1 to +5. As used herein, the terms “nitrogen oxides” and ‘oxides of nitrogen’ and ‘NOₓ’ mean a gas having one or more of the following gases, all of which contain nitrogen and oxygen in varying amounts: nitric oxide (NO) nitrogen dioxide (NO₂), nitrogen trioxide (NO₃), dinitrogen trioxide (N₂O₃), dinitrogen tetroxide (N₂O₄), dinitrogen pentoxide (N₂O₅) and nitrous oxide (N₂O). As used herein, the phrase “nitric oxide precursor” means a compound or composition capable of producing or releasing NO, NO₂, and NOₓ.

In view of the foregoing, the inventors contemplate utilizing the composite article 10 and the composition to form nitric oxide for a variety of uses. Non-limiting examples of suitable uses of the composite article or the composition capable of forming nitric oxide include food packaging for preserving foodstuff (e.g., meats, fruits, vegetables, cheeses, ingredients thereof, and the like); components of vehicles for sanitizing vehicles (e.g., headliners, seat cushion liners, carpet liners, and the like); hygienic containers for sanitizing hygiene devices (e.g., toothbrushes, mouth/bite guards, CPAP masks, facemasks, and the like); medical device containers for sanitizing medical instruments (e.g., stethoscopes, otoscopes, and the like), medical devices (e.g., portable ultrasound device, communication devices, and the like); components of devices exposed to moisture for resisting growth of mold or mildew (e.g., washing machines, boat compartments, and the like); liners for sporting equipment bags for sanitizing sporting equipment (e.g., shoes, hockey equipment, ski equipment, facemasks, googles, helmets, and the like); and sealants for resisting growth of mold or mildew (e.g., window sealants, shower/bath sealants, and the like).

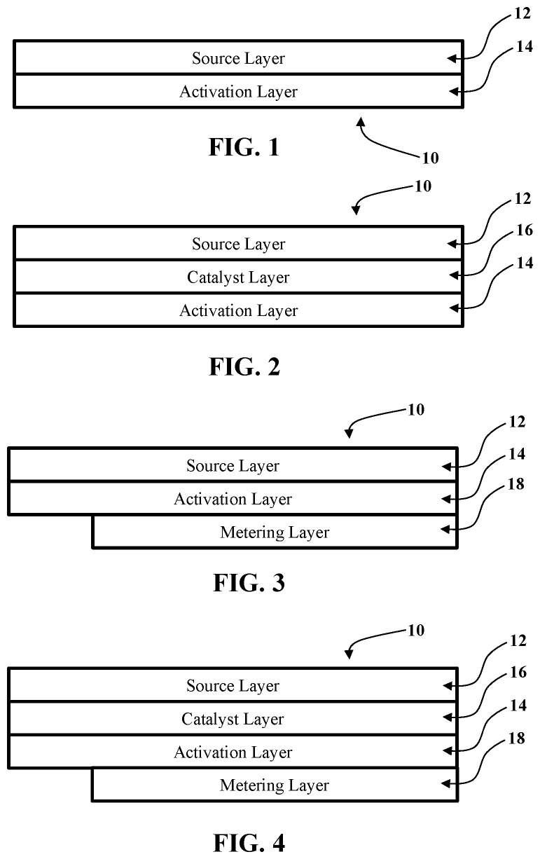

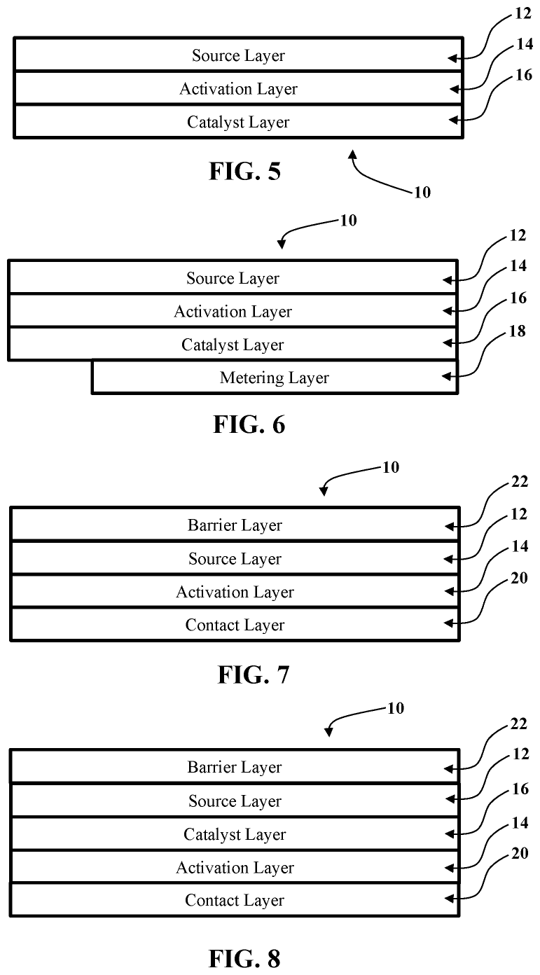

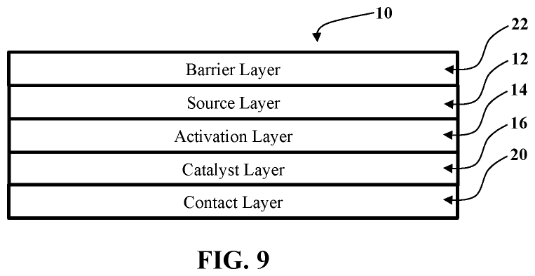

FIGS. 1-9 illustrate non-limiting embodiments of the composite article 10. The composite article 10 includes, consists essentially of, or consists of, a source layer 12 and an activation layer 14 disposed overlying the source layer 12. The source layer 12 includes, consists essentially of, consists of, or is, a nitric oxide precursor. The activation layer 14 includes, consists essentially of, consists of, or is, a thiol-containing compound. The nitric oxide precursor and the thiol-containing compound are capable of reacting in the presence of a solvent (e.g., water) to form nitric oxide.

As introduced above, the composition is also provided herein. The composition includes, consists essentially of, consists of, or is, a source portion and an activation portion. The source portion includes, consists essentially of, consists of, or is, the nitric oxide precursor. The activation portion includes, consists essentially of, consists of, or is, the thiol-containing compound. Once again, the nitric oxide precursor and the thiol-containing compound are capable of reacting in the presence of a solvent (e.g., water) to form nitric oxide.

In particular, in certain embodiments, the nitric oxide precursor and the thiol-containing compound are capable of reacting in the presence of the solvent to form a nitrosothiol which is generally unstable and capable of decomposing to form the nitric oxide. To this end, in exemplary embodiments, the solvent (e.g., water moisture from the environment) is absorbed into the composite article 10 and solubilizes at least the nitric oxide precursor. The nitric oxide precursor is then mobilized in the solvent and moves through the source layer 12 to the activation layer 14. However, it is to be appreciated that the solvent may solubilize the thiol-containing compound thereby mobilizing the thiol-containing compound in the solvent such that the thiol-containing compound moves through the activation layer 14 to the source layer 12. The nitric oxide precursor and the thiol-containing compound then react to form an unstable S-nitrosothiol in situ that rapidly decomposes to release nitric oxide. The nitric oxide then moves out of composite article 10 to sanitize the area proximate the composite article 10, such as via interaction with microorganisms in this area. Importantly, in various embodiments, the nitric oxide is capable of being formed (a) at a temperature of from −50° C. to 150° C., (b) in the presence or absence of visible light, or (c) a combination thereof.

As introduced above, the source layer 12 is overlying the activation layer 14. It is to be appreciated that the term “overlying” is not to be construed as limiting the composite article in any way, such as by limiting the composite article to a particular configuration or method of forming. Furthermore, the source layer 12 may be overlying or disposed on any portion(s) of the activation layer 14, as will be understood by those of skill in the art. For example, the source layer 12 may be overlying or disposed on one face or side of the activation layer 14. Furthermore, for example, the source layer 12 may be disposed on only a portion of the one face or side of the activation layer 14. In certain embodiments, the source layer 12 and activation layer 14 are in direct contact. In other words, there is not an intervening layer between the source layer 12 and activation layer 14. In other embodiments, the composite article 10 includes other layers in variety of configurations relative to the source layer 12 and activation layer 14, as will be described in greater detail below.

The nitric oxide precursor of the source layer 12 may be associated with the source layer 12 in any manner known in the art. In some embodiments, the nitric oxide precursor is substantially homogeneously disposed throughout the source layer 12. In other embodiments, the nitric oxide precursor is contained within the source layer 12 at a gradient (e.g., a higher concentration proximate at least one of the faces of the source layer 12 or a higher concentration proximate the middle of the source layer 12). In still other embodiments, the nitric oxide precursor is a coating relative to the source layer 12. In yet other embodiments, the nitric oxide precursor is the source layer 12.

There is a wide variety of possible nitric oxide precursors that can be used, depending on the design constraints of the desired application of the composite article 10 or composition. The nitric oxide precursor can include, but is not limited to, SNAP-PDMS and other nitric oxide donating polymers that use different nitric oxide moieties and different polymer base materials. The nitric oxide donors can be covalently linked to the polymer or blended into the polymer. Discrete nitric oxide donors can also be used in solid, liquid or gel forms. Non-limiting examples of this include one or more of S-nitroso-N-acetyl-D-penicillamines (SNAP), nitrite, S-nitrosocysteine, S-nitrosoglutathione, diazeniumdiolate compounds, enzymatic generation of NO from arginine, or organitrites, biological sources such a macrophage generation, etc. Non-limiting examples of suitable S-nitroso-N-acetyl-D-penicillamines and other photosensitive S-nitrosothiols covalently attached to polymers are described in U.S. Pat. No. 9,884,943 B2 and International Publication No. WO 2020/018488 A1, which are incorporated by reference in their entirety. Non-limiting examples of other suitable nitric oxide precursors are described in U.S. Pat. App. Pub. No. 2021/0220523 A1, which is incorporated by reference in its entirety.

Other non-limiting examples of nitric oxide precursors include one or more of gas phase delivery from polymers, acidified nitrite or nitrate, nitric oxide donating molecules such as diazeniumdiolates, nitrosothiols, nitrosyl compounds, or other methods of NO generation such as enzymatic production of nitric oxide, chemical production of nitric oxide from ascorbic acid or metal catalysis, electrochemical production of nitric oxide, photolytic cleavage of bonds to release nitric oxide, direct delivery of nitric oxide gas, etc.

In certain embodiments, the nitric oxide precursor may include a nitrite. The nitrite may be selected from the group of sodium nitrite, calcium nitrite, potassium nitrite, tetrabutylammonium nitrite, dicyclohexylammonium nitrite, butylnitrite, isobutylnitrite, t-butylnitrite, amylnitrite, pentylnittrite, nitrite salts, ion paired nitrite, silver nitrite, zinc nitrite, iron nitrite, copper nitrite, transition metal-nitrite compounds, and combinations thereof.

The nitric oxide precursor may be present in the source layer 12 in any amount suitable for forming nitric oxide. In certain embodiments, the nitric oxide precursor is present in the source layer 12 in an amount of from about 0.01 to about 100, optionally from about 0 to about 99, or optionally from about 0.01 to about 99, wt. % based on a total weight of the source layer 12. Additional subranges of the preceding endpoints, and other points in-between, are also contemplated.

Referring back to the source layer 12, the source layer 12 may be in a solid or semi-solid form. In various embodiments, the source layer 12 is in a solid form. The source layer 12 may be thermoplastic or thermoset. The source layer 12 may be of any shape and dimension, which are each typically selected based on the intended use of the composite article 10. The source layer 12 may have an average thickness of from about 0.01 to about 100, optionally from about 0.1 to about 40, or optionally from about 0.01 to about 4, mil(s).

In various embodiments, the source layer 12 includes a carrier material. The carrier material may be thermoplastic or thermoset. Non-limiting examples of suitable thermoplastic materials include polyvinyl chloride (“PVC”), polyethylene terephthalate (“PET”), polyethylene terephthalate glycol-modified (“PETG”), polypropylene (“PP”), polyethylene (“PE”), polyamide, such as nylon, and combinations thereof. Non-limiting examples of suitable thermoset materials include UV curable materials, heat curable materials, chemical curable materials, such as free radical, room temperature curable materials, and cold cured materials.

In certain embodiments, the carrier material of the source layer 12 may be formed from a cellulose, a polyvinyl chloride, a polyurethane, a carbosil, a polydimethylsiloxane, an acrylic polymer, a polyester, a poly(lactic acid), a poly(lactic-co-glycolic acid), poly(vinyl acetate), ethylene vinyl acetate, tecothane, pellethane, a hydrogel, a polytetrafluoroethylene, a copolymer thereof, or combinations thereof. In one exemplary embodiment, the carrier material is formed from a cellulose. In another exemplary embodiment, the carrier material is formed from a hydrogel selected from the group of a polymacron, a polyacrylamide, a collagen, an agarose, a hyaluronic acid, a poly(organophosphazenes), a chitosan, a poly(ethylene glycol), poly(vinyl alcohol), and combinations thereof. Non-limiting examples of suitable hydrogels are described in a journal article titled “S-Nitrosothiol Detection via Amperometric Nitric Oxide Sensor with Surface Modified Hydrogel Layer Containing Immobilized Organoselenium Catalyst” cited as Langmuir 2006, 22, 25, 10830-10836, which is incorporated by reference in its entirety.

In other exemplary embodiments, the carrier material of the source layer 12 includes a support. The support may include polytetrafluoroethylene in the form of a mesh or a fiber. However, it is to be appreciate that any type of material and form of support may be utilized for the support. In these and other embodiments, the carrier material further includes a material disposed within the support and formed from a cellulose, a polyvinyl chloride, a polyurethane, a carbosil, a polydimethylsiloxane, an acrylic polymer, a polyester, a poly(lactic acid), a poly(lactic-co-glycolic acid), poly(vinyl acetate), ethylene vinyl acetate, tecothane, pellethane, a hydrogel, another polytetrafluoroethylene, a copolymer thereof, or combinations thereof.

In certain embodiments, the source layer 12 has a permeability in an amount of at least 0.001, optionally at least 0.1, optionally at least, optionally at least 1, or optionally at least 5 g/(m·s·Pa) in accordance with ASTM E2945-14(2021) for permitting movement of at least one of the nitric oxide precursor or the solvent through the source layer 12 to the activation layer 14.

In various embodiments, the source layer 12 has a moisture content of less than about 20, less than about 15, less than about 10, less than about 5, less than about 1, or approaching 0, parts by weight, based on 100 parts by weight of the source layer 12. Too much moisture prior to use of the composite article 10 may prematurely allow movement of the nitric oxide precursor through the source layer 12 to the activation layer 14 thereby initiating the formation of nitric oxide.

Referring now to the thiol-containing compound of the activation layer 14, the thiol-containing compound may be associated with the activation layer 14 in any manner known in the art. In some embodiments, the thiol-containing compound is substantially homogeneously disposed throughout the activation layer 14. In other embodiments, the thiol-containing compound is contained within the activation layer 14 at a gradient (e.g., a higher concentration proximate at least one of the faces of the activation layer 14 or a higher concentration proximate the middle of the activation layer 14). In still other embodiments, the thiol-containing compound is a coating relative to the activation layer 14. In yet other embodiments, the thiol-containing compound is the activation layer 14.

There is a wide variety of possible thiol-containing compounds that can be used, depending on the design constraints of the desired application of the composite article 10 or composition. The thiol-containing compound can include, but is not limited to, one or more of 1,2-ethane dithiol, 2,3-dimercaptopropanol, pyrithione, dithioerythritol, 3,4-dimercaptotoluene, 2,3-butanedithiol, 1,3-propanedithiol, 2-hydroxypropane thiol, 1-mercapto-2-propanol, dithioerythritol and dithiothreitol. Other exemplary thiol-containing compounds include alpha-lipoic acid, methanethiol (CH₃SH [m-mercaptan]), ethanethiol (C₂H₅SH [e-mercaptan]), 1-propanethiol (C₃H₇SH [n-P mercaptan]), 2-propanethiol (CH₃CH(SH)CH₃ [₂C₃ mercaptan]), butanethiol (C₄H₉SH ([n-butyl mercaptan]), tert-butyl mercaptan (C(CH₃)₃SH [t-butyl mercaptan]), pentanethiols (C₅H₁SH [pentyl mercaptan]), coenzyme A, lipoamide, glutathione, cysteine, cystine, 2-mercaptoethanol, dithiothreitol, dithioerythritol, 2-mercaptoindole, transglutaminase, (11-mercaptoundecyl)hexa(ethylene glycol), (11-mercaptoundecyl)tetra(ethylene glycol), (11-mercaptoundecyl)tetra(ethylene glycol) functionalized gold nanoparticles, 1,1′,4′,1″-terphenyl-4-thiol, 1,11-undecanedithiol, 1,16-hexadecanedithiol, 1,2-ethanedithiol, 1,3-propanedithiol, 1,4-benzenedimethanethiol, 1,4-butanedithiol, 1,4-butanedithiol diacetate, 1,5-pentanedithiol, 1,6-hexanedithiol, 1,8-octanedithiol, 1,9-nonanedithiol, adamantanethiol, 1-butanethiol, 1-decanethiol, 1-dodecanethiol, 1-heptanethiol, 1-heptanethiol purum, 1-hexadecanethiol, 1-hexanethiol, 1-mercapto-(triethylene glycol), 1-mercapto-(triethylene glycol) methyl ether functionalized gold nanoparticles, 1-mercapto-2-propanol, 1-nonanethiol, 1-octadecanethiol, 1-octanethiol, 1-octanethiol, 1-pentadecanethiol, 1-pentanethiol, 1-propanethiol, 1-tetradecanethiol, 1-tetradecanethiol purum, 1-undecanethiol, 11-(1H-pyrrol-1-yl)undecane-1-thiol, 11-amino-1-undecanethiol hydrochloride, 11-bromo-1-undecanethiol, 11-mercapto-1-undecanol, 11-mercapto-1-undecanol, 11-mercaptoundecanoic acid, 11-mercaptoundecanoic acid, 11-mercaptoundecyl trifluoroacetate, 11-mercaptoundecylphosphoric acid, 12-mercaptododecanoic acid, 12-mercaptododecanoic acid, 15-mercaptopentadecanoic acid, 16-mercaptohexadecanoic acid, 16-mercaptohexadecanoic acid, 1H, 1H,2H,2H-perfluorodecanethiol, 2,2′-(ethylenedioxy)diethanethiol, 2,3-butanedithiol, 2-butanethiol, 2-ethylhexanethiol, 2-methyl-1-propanethiol, 2-methyl-2-propanethiol, 2-phenylethanethiol, 3,3,4,4,5,5,6,6,6-nonafluoro-1-hexanethiol purum, 3-(dimethoxymethylsilyl)-1-propanethiol, 3-chloro-1-propanethiol, 3-mercapto-1-propanol, 3-mercapto-2-butanol, 3-mercapto-N-nonylpropionamide, 3-mercaptopropionic acid, 3-mercaptopropyl-functionalized silica gel, 3-methyl-1-butanethiol, 4,4′-bis(mercaptomethyl)biphenyl, 4,4′-dimercaptostilbene, 4-(6-mercaptohexyloxy)benzyl alcohol, 4-cyano-1-butanethiol, 4-mercapto-1-butanol, 6-(ferrocenyl)hexanethiol, 6-mercapto-1-hexanol, 6-mercaptohexanoic acid, 8-mercapto-1-octanol, 8-mercaptooctanoic acid, 9-mercapto-1-nonanol, biphenyl-4,4′-dithiol, butyl 3-mercaptopropionate, copper(I) 1-butanethiolate, cyclohexanethiol, cyclopentanethiol, decanethiol functionalized silver nanoparticles, dodecanethiol functionalized gold nanoparticles, dodecanethiol functionalized silver nanoparticles, hexa(ethylene glycol)mono-11-(acetylthio)undecyl ether, mercaptosuccinic acid, methyl 3-mercaptopropionate, octanethiol functionalized gold nanoparticles, PEG dithiol, S-(11-bromoundecyl)thioacetate, S-(4-cyanobutyl)thioacetate, thiophenol, triethylene glycol mono-11-mercaptoundecyl ether, trimethylolpropane tris(3-mercaptopropionate), [11-(methylcarbonylthio)undecyl]tetra(ethylene glycol), m-carborane-9-thiol, p-terphenyl-4,4″-dithiol, tert-dodecylmercaptan, or tert-nonyl mercaptan.

In certain embodiments, the thiol-containing compound includes a cysteine or derivative thereof, a thiol-derivatized polymer or filler, or a combination thereof. In embodiments when the cysteine or derivative thereof is utilized, the cysteine or derivative thereof may be selected from the group of cysteine, glutathione, acetyl cysteine, penicillamine, acetylpenicillamine, S-nitroso-n-acetylpenicillamine, bucillamine, and combinations thereof. It is to be appreciated that the thiol-containing compound may be included as part of a peptide, a polymer, a copolymer, or other macromolecules. In embodiments when the cysteine or derivative thereof is utilized as part of a peptide, the peptide may include any combination of amino acids so long as the peptide includes the cysteine or derivative thereof as at least one of the constituents of the peptide. Non-limiting examples of suitable cysteines or derivatives thereof are described in a journal article titled “S-Nitrosothiol Detection via Amperometric Nitric Oxide Sensor with Surface Modified Hydrogel Layer Containing Immobilized Organoselenium Catalyst” cited as Langmuir 2006, 22, 25, 10830-10836, which is incorporated by reference in its entirety.

The thiol-containing compound may be present in the activation layer 14 in any amount suitable for forming nitric oxide. In certain embodiments, the thiol-containing compound is present in the activation layer 14 in an amount of from about 0.1 to about 100, optionally from about 5 to about 10, or optionally from about 20 to about 50, wt. % based on a total weight of the activation layer 14. Additional subranges of the preceding endpoints, and other points in-between, are also contemplated.

Referring back to the activation layer 14, the activation layer 14 may be in a solid or semi-solid form. In various embodiments, the activation layer 14 is in a solid form. The activation layer 14 may be thermoplastic or thermoset. The activation layer 14 may be of any shape and dimension, which are each typically selected based on the intended use of the composite article 10. The activation layer 14 may have an average thickness of from about 0.01 to about 100, optionally from about 0.1 about 2, or optionally from about 1 to about 4, mil(s). In various embodiments, the activation layer 14 includes the carrier material described above for the source layer 12. However, it is to be appreciated that the carrier material may be different for each of the layers described herein.

In certain embodiments, the activation layer 14 has a permeability in an amount of at least 0.001, optionally at least 0.1, optionally at least 1, or optionally at least 5 g/(m·s·Pa) in accordance with ASTM E2945-14(2021) for permitting movement of at least one of the nitric oxide precursor or the solvent through the activation layer 14 or, in alternative embodiments, at least one of the thiol-containing compound or the solvent through the activation layer 14 to the source layer 12.

In various embodiments, the activation layer 14 has a moisture content of less than about 20, less than about 15, less than about 10, less than about 5, less than about 1, or approaching 0, parts by weight, based on 100 parts by weight of the activation layer 14. Too much moisture prior to use of the composite article 10 may prematurely allow movement of at least one of the nitric oxide precursor or the thiol-containing compound through the source layer 12 to the activation layer 14, or, in alternative embodiments, through the activation layer 14 to the source layer 12, thereby initiating the formation of nitric oxide.

Moving on, the composite article 10 or the composition may further include a catalyst. The inventors contemplate that the catalyst may modulate (e.g., increase reaction rate, decrease reaction rate, etc.) the reduction of S-nitrosothiol to nitric oxide. The catalyst may include a transition metal, a non-metal, or a combination thereof. However, it is to be appreciated that the catalyst may include any compound that can modulate the reduction of S-nitrosothiol to nitric oxide known in the art. In some embodiments, the catalyst includes a transition metal selected from the group of copper (Cu), zinc (Zn), silver (Ag), gold (Au), lead (Pb), platinum (Pt), iron (Fe), magnesium (Mg), manganese (Mn), cobalt (Co), nickel (Ni), and combinations thereof. Non-limiting examples of suitable copper (Cu) catalysts are described in International Publication No. WO 2005/094913 A1, U.S. Pat. No. 8,168,423 B2, and a journal article titled “Spontaneous Catalytic Generation of Nitric Oxide from S-Nitrosothiols at the Surface of Polymer Films Doped with Lipophilic Copper(II) Complex” cited as J. Am. Chem. Soc. 2003, 125, 32, 9552-9553, which are incorporated by reference in their entirety. In exemplary embodiments, the catalyst is zinc chloride.

In other embodiments, the catalyst includes a non-metal selected from the group of selenium (Se), tellurium, (Te), an organometal compound, and combinations thereof. In exemplary embodiments, the catalyst is selenium, an organoselenium, or a combination thereof. Non-limiting examples of suitable organoselenium catalysts are described in a journal article titled “S-Nitrosothiol Detection via Amperometric Nitric Oxide Sensor with Surface Modified Hydrogel Layer Containing Immobilized Organoselenium Catalyst” cited as Langmuir 2006, 22, 25, 10830-10836, which is incorporated by reference in its entirety.

The catalyst may be associated with any layer of the composite article 10 in any manner known in the art. In some embodiments, the catalyst is substantially homogeneously disposed throughout at least one of the layers. In other embodiments, the catalyst is contained within at least one of the layers at a gradient (e.g., a higher concentration proximate at least one of the faces of the layer or a higher concentration proximate the middle of the layer). In still other embodiments, the catalyst is a coating relative to at least one of the layers. In yet other embodiments, the catalyst is its own layer.

The catalyst may be present in the source layer 12, the activation layer 14, a catalytic layer 16 (described in greater detail below), or combinations thereof. In certain embodiments, the catalyst is present in at least one of the source layer 12, the activation layer 14, or the catalyst layer 0.01, in an amount of from about 0.1 to about 100, optionally from about 0.02 to about 0.5, or optionally from about 1 to about 10, wt. % based on a total weight of each of the layers containing the catalyst. Additional subranges of the preceding endpoints, and other points in-between, are also contemplated.

The composite article 10 may further include the catalytic layer 16. The catalytic layer 16 may be overlying, on, or disposed between any of the layers of the composite article 10. In certain embodiments, the catalytic layer 16 may be (a) disposed between the source layer 12 and the activation layer 14, (b) disposed on the source layer 12 and optionally spaced from the activation layer 14, (c) disposed on the activation layer 14 and optionally spaced from the source layer 12, or (d) combinations thereof.

The catalytic layer 16 may be in a solid or semi-solid form. In various embodiments, the catalytic layer 16 is in a solid form. The catalytic layer 16 may be thermoplastic or thermoset. The catalytic layer 16 may be of any shape and dimension, which are each typically selected based on the intended use of the composite article 10. The catalytic layer 16 may have an average thickness of from about 0.01 to about 100, optionally from about 0.1 to about 1, or optionally from about 1 to about 5 mil(s). In various embodiments, the catalytic layer 16 includes the carrier material described above for the source layer 12. However, it is to be appreciated that the carrier material may be different for each of the layers described herein.

In certain embodiments, the catalytic layer 16 has a permeability in an amount of at least 0.001, optionally at least 0.1, optionally at least, optionally at least 1, or optionally at least 5 g/(m·s·Pa) in accordance with ASTM E2945-14(2021) for permitting movement of the components of the composite material XX through the catalytic layer 16.

Moving on, the composite article 10 may further include a metering layer 18 capable of modulating the movement of at least one of the nitric oxide precursor or the solvent to or through the activation layer 14, or, in the alternative, modulating the movement of at least one of the thiol-containing compound or the solvent to or through the source layer 12. The metering layer 18 may be overlying, on, or disposed between any of the layers of the composite article 10. In certain embodiments, the metering layer 18 may be (a) disposed between the source layer 12 and the activation layer 14, (b) disposed on the source layer 12 and optionally spaced from the activation layer 14, (c) disposed on the activation layer 14 and optionally spaced from the source layer 12, or (d) combinations thereof. In exemplary embodiments, the metering layer 18 is disposed on the activation layer 14 and spaced from the source layer 12. In these and other embodiments, the metering layer 18 only partially overlies the activation layer 14 such that a portion of the activation layer 14 is free of the metering layer 18.

The metering layer 18 may be in a solid or semi-solid form. In various embodiments, the metering layer 18 is in a solid form. The metering layer 18 may be thermoplastic or thermoset. The metering layer 18 may be of any shape and dimension, which are each typically selected based on the intended use of the composite article 10. The metering layer 18 may have an average thickness of from about 0.01 to about 100, optionally from about 0.1 to about 1, or optionally from about 1 to about 5 mil(s). In various embodiments, the metering layer 18 includes the carrier material described above for the source layer 12. However, it is to be appreciated that the carrier material may be different for each of the layers described herein.

The metering layer may have a permeability different than the permeability of at least one of the source layer 12 or the activation layer 14. In certain embodiments, the metering layer 18 has a permeability in an amount of at least 0.001, optionally at least 0.1, optionally at least, optionally at least 1, or optionally at least 5 g/(m·s·Pa) in accordance with ASTM E2945-14(2021), but less than the permeability of at least one of the source layer 12 or the activation layer 14 for permitting modulated movement of the components of the composite article 10 through the metering layer 18.

The composite article 10 or the composition may further include a variety of additives, including, but not limited to, ascorbate, reducing equivalents, oxidizing equivalents, acids, bases, pH buffers, ionophores, enzymes, any agent that will impact the formation and stability of thiols (e.g., including disulfide formation or breaking of disulfide bonds), nitrosothiols (e.g., acids/bases, ion mobility, gas permeability, reaction/buffering of NO gas), plasticizers, surfactants, colorants, fillers, or combinations thereof.

The plasticizer may include a plasticizer that may be used to modify various characteristics including, but not limited to, permeability, modifying hydrophobicity, tensile strength, elongation, and the like. The plasticizer includes, but is not limited to, phthalates, trimellitates, benzoates, adipates, sebacates, maleates, citrates, epoxidized vegetable oils, sulfonamides, organophosphates, glycols/polyethers, polymeric plasticizers and polybutenes, or combinations thereof. However, it is to be appreciated that the plasticizer may include any other plasticizer understood in the art so long as the plasticizer is compatible with the components of the composite article 10 or the composition.

The plasticizer may be an ester plasticizer. Examples of suitable ester plasticizers include, but are not limited to, dioctyl phthalate (DOP), n-hexyl-n-decyl phthalate (NHDP), n-octyl-decyl phthalate (NODP), di(isononyl) phthalate (DINP), di(isodecyl)phthalate (DIDP), diundecyl phthalate (DUP), di(isotridecyl)phthalate (DTDP), di-2-ethylhexyl adipate (DOA), di-n-octyl-n-decyl adipate (DNODA), diisononyl adipate (DINA), di-2-ethylhexyl azelate (DOZ), di-2-ethylhexyl sebacate (DOS), trioctyl trimellitate (TOTM), trioctyl phosphate (TOP), tricresyl phosphate (TCP), aliphatic polyester plasticizer, aliphatic polyol plasticizer, or combinations thereof. In certain embodiments, the plasticizer component includes trioctyl trimellitate (TOTM). It is to be appreciated the plasticizer may include any phthalate known in the art so long as it is compatible with the composite article 10 or the composition.

The surfactant component may include anionic surfactants, non-ionic surfactants, cationic surfactants, Zwitterionic surfactants, or combinations thereof. However, it is to be appreciated that the surfactant component may include any other surfactant understood in the art so long as the surfactant is compatible with the components of the composite article 10 or the composition.

Examples of suitable anionic surfactants include, but are not limited to, fatty alcohol sulphates, alkylphenol sulphates, fatty alcohol ether sulphates, fatty alcohol ether sulphates, alkylphenol ether sulphates, alkylbenzene sulphonic acid, alkyl ether carboxylic acid and salts thereof, alkyl sulphosuccinates, alkyl sulphosuccinates, phosphate esters, α-olefin sulphonates, or combinations thereof. Examples of suitable non-ionic surfactants include, but are not limited to, alcohol ethoxylates, alkylphenol ethoxylates, polyethylene oxide/polyethylene oxide block copolymers, polyvinyl alcohol, polyvinyl pyrrolidone, sorbitan fatty acid esters, sorbitan ester ethoxylates, or combinations thereof. Examples of suitable cationic surfactants includes, but are not limited to, alkyl dimethylamines, quaternary ammonium compounds, or combinations thereof. In certain embodiments, the surfactant component includes a nonionic surfactant. The nonionic surfactant may include an acetylene glycol surfactant, 2-ethylhexanol, or a combination thereof.

The filler may include any filler that may be used for various objectives including, but not limited to, cost control, rheology control, lubricity modification, as well as to prevent seizing or galling. The filler component may include an inorganic filler. Examples of suitable inorganic fillers include, but are not limited to, powdered nickel, copper, zinc, and aluminum. Suitable mineral fillers include, but are not limited to, talc, calcium carbonate, silicates such as mica, wollastonite, titanium dioxide, quarts, fumed silica precipitated silica, graphite, boron nitride, or combinations thereof.

Other components that may be present in the composite article 10 or the composition include minor amounts of antioxidants, inhibitors, defoamers, dispersing aids, heat stabilizers, UV stabilizers, and the like, such as one or more components described in U.S. Patent App. Pub. No. 2004/0258922 A1, in U.S. Pat. No. 9,404,015 B2, and U.S. Pat. No. 10,214,668 B2, the disclosures of which are incorporated herein by reference in their entirety. In various embodiments, one or more of such additives are individually present in the composite article 10 or the composition in an amount less than about 5 wt. % based on a total weight of the composite article 10 or the composition.

A food packaging article capable of forming nitric oxide to preserve foodstuff is also provided herein. The food packaging article includes the source layer 12 and the activation layer 14 overlying the source layer 12, as described above. The food packaging article further includes a contact layer 20 overlying the activation layer 14. The contact layer may have a permeability in an amount of at least 0.001, optionally at least 0.1, optionally at least, optionally at least 1, or optionally at least 5 g/(m·s·Pa), in accordance with ASTM E2945-14(2021) for permitting movement of nitric oxide therethrough. The contact layer 20 may be substantially impermeable to ions. The food packaging article further may include a barrier layer 22. The source layer 12 is disposed on the barrier layer 22 and the barrier layer 22 is spaced from the activation layer 14. The barrier layer 22 has a permeability in an amount of less than 0.01 g/(m·s·Pa) in accordance with ASTM E2945-14(2021) to prevent movement of nitric oxide therethrough. The food packaging article may be in the form of a rigid container, wrapper, bag, bottle, or tube. In various embodiments, poultry contained within the food packaging article exhibits a decrease in spoilage after 96 hours at 23° C. as compared to poultry contained with a container free of at least one of the nitric oxide precursor or the thiol-containing compound.

A vehicle headliner of a vehicle capable of forming nitric oxide to sanitize the vehicle is also provided. The vehicle headliner includes the source layer 12 and the activation layer 14, as described above. The vehicle headliner further includes a fabric layer overlying the activation layer 14.

A washing machine component capable of forming nitric oxide to resist growth of mold or mildew is also provided. The washing machine component includes the source portion and the activation portion. In certain embodiments, the washing machine component is a gasket or a liner.

A sealant composition capable of forming nitric oxide to resist growth of mold or mildew is also provided. The sealant composition includes the source portion and the activation portion. The sealant composition further includes a sealant material. In certain embodiments, the sealant material is selected from the group of a silicone, an epoxy, a polyurethane, a polysulfide, a latex, and combinations thereof.

The composite article 10 can be formed utilizing conventional techniques understood in the art. In an exemplary method of forming, the method includes the step of providing a first carrier material and a second carrier material. The method further includes the step of combining the nitric oxide precursor and water to form a first solution. The method further includes the step of combining the thiol-containing compound and water to form a second solution. The method further includes the step of disposing the applying the first solution to the first carrier material to form the source layer 12. The method further includes the step of applying the second solution to the second carrier material to form the activation layer 14. The method further includes the step of disposing the activation layer 14 on the source layer 12 to form the composite article 10.

The carrier materials may be formed by various methods understood in the art. For example, the carrier materials can be extruded, cast, laminated, etc. The solutions may be applied to the carriers by various methods understood in the art. For example, the solutions can be sprayed onto the carriers, the carriers may be submerged into the solutions, the carriers and the solutions may be combined and then extruded or cast to form the layers. It is to be appreciated any other method known in the art for forming composite articles may be utilized so long as the methods are compatible with the components of the composite article 10.

The composition can be formed utilizing conventional techniques understood in the art. In an exemplary method of forming, the method includes the step of combining a carrier material, the nitric oxide precursor, and the thiol-containing compound to form the composition. In embodiments when the composition is a sealant, the step of combining may further include the sealant material.

### EXAMPLES

The following examples are included to demonstrate various embodiments as contemplated herein. It should be appreciated by those of skill in the art that the techniques disclosed in the examples which follow represent techniques discovered by the inventor(s) to function well in the practice of the invention, and thus can be considered to constitute desirable modes for its practice. However, those of skill in the art should, in light of the present disclosure, appreciate that many changes can be made in the specific embodiments which are disclosed and still obtain a like or similar result without departing from the spirit and scope of the invention. All percentages are in wt. % and all measurements are conducted at 23° C. unless indicated otherwise.

### Exemplary Formulations of the Composite Article

Provided below is an exemplary formulation of the composite article.

TABLE I
Formulations

| | | | | | | |
|---|---|---|---|---|---|---|
| | Ex. Form. | Ex. Form. | Ex. Form. | Ex. Form. | Ex. Form. | Ex. Form. |
| Component | I (wt. %) | II (wt. %) | III (wt. %) | IV (wt. %) | V (wt. %) | VI (wt. %) |

Source Layer

| | | | | | | |
|---|---|---|---|---|---|---|
| Carrier I | 80 to 99 | — | 80 to 99 | — | 80 to 99 | — |
| Carrier II | — | 80 to 99 | — | 80 to 99 | — | 80 to 99 |
| Nitric Oxide | 1 to 20 | 1 to 20 | 1 to 20 | 1 to 20 | 1 to 20 | 1 to 20 |
| Precursor I | | | | | | |
| TOTAL | 100 | 100 | 100 | 100 | 100 | 100 |

Activation Layer

| | | | | | | |
|---|---|---|---|---|---|---|
| Carrier I | 80 to 99 | 80 to 99 | — | — | 80 to 99 | — |
| Carrier II | — | — | 80 to 99 | 80 to 99 | — | 80 to 99 |
| Thiol- | 1 to 20 | 1 to 20 | 1 to 20 | 1 to 20 | 1 to 20 | 1 to 20 |
| Containing | | | | | | |
| Compound I | | | | | | |
| TOTAL | 100 | 100 | 100 | 100 | 100 | 100 |

Catalyst Layer

| | | | | | | |
|---|---|---|---|---|---|---|
| Carrier I | — | — | — | — | 80 to 99 | — |
| Carrier II | — | — | — | — | — | 80 to 99 |
| Catalyst I | — | — | — | — | 1 to 20 | 1 to 20 |
| TOTAL | — | — | — | — | 100 | 100 |

Carrier I is a cellulose which is commercially available.
Carrier II is an ethylene vinyl acetate which is commercially available.
Nitric Oxide Precursor I is a sodium nitrite which is commercially available.
Thiol-Containing Compound I is a glutathione which is commercially available.
Catalyst I is zinc chloride which is commercially available.

### Exemplary Formulations of the Composition

Provided below is an exemplary formulation of the composition.

TABLE II
Formulations

| | Ex. Form. | Ex. Form. | Ex. Form. | Ex. Form. | Ex. Form. | Ex. Form. |
|---|---|---|---|---|---|---|
| Component | VII (wt. %) | VIII (wt. %) | IX (wt. %) | X (wt. %) | XI (wt. %) | XII (wt. %) |
| Carrier I | 80 to 99 | — | 80 to 99 | — | 80 to 99 | — |
| Carrier II | — | 80 to 99 | — | 80 to 99 | — | 80 to 99 |
| Nitric Oxide | 1 to 20 | 1 to 20 | 1 to 20 | 1 to 20 | 1 to 20 | 1 to 20 |
| Precursor I | | | | | | |
| Thiol- | 1 to 20 | 1 to 20 | 1 to 20 | 1 to 20 | 1 to 20 | 1 to 20 |
| Containing | | | | | | |
| Compound I | | | | | | |
| Catalyst I | — | — | — | — | 1 to 20 | 1 to 20 |
| TOTAL | 100 | 100 | 100 | 100 | 100 | 100 |

Carrier I is a cellulose which is commercially available.
Carrier II is an ethylene vinyl acetate which is commercially available.
Nitric Oxide Precursor I is a sodium nitrite which is commercially available.
Thiol-Containing Compound I is a glutathione which is commercially available.
Catalyst I is zinc chloride which is commercially available.

### Example I: Composite Article

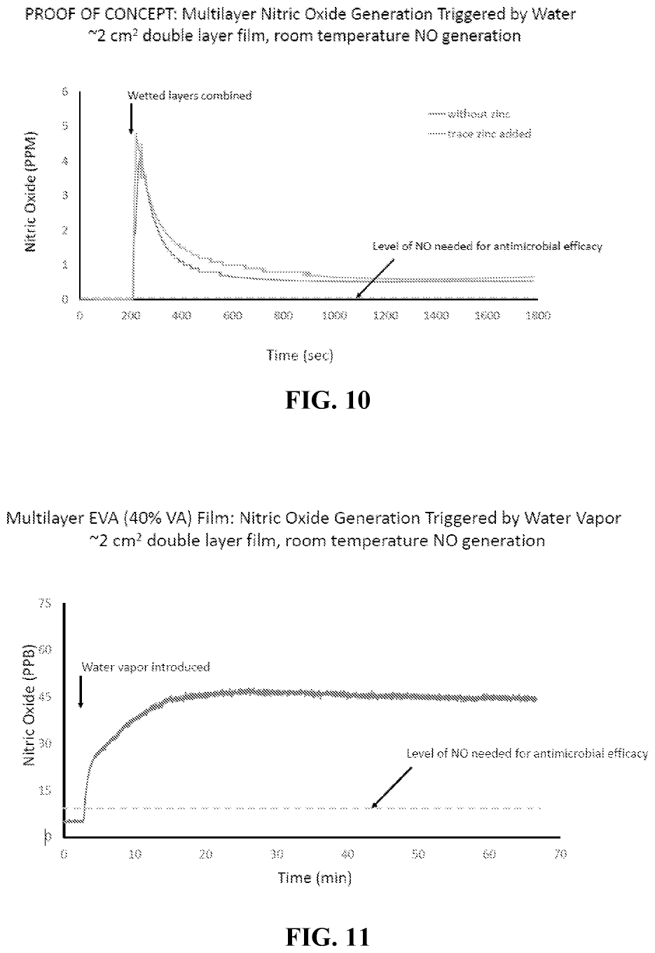

A cellulose carrier was saturated with an aqueous solution containing sodium nitrite. Another cellulose carrier was saturated with an aqueous solution containing reduced glutathione. The two layers were stacked on top of each other to form the composite article. The composite article was then exposed to water and generated nitric oxide by formation of S-nitrosoglutathione, which then rapidly decomposed to release nitric oxide (see FIG. 10). Alone, neither layer generated nitric oxide. Further, without the exposure to water, the layers together did not generate nitric oxide. However, when both layers were in contact with each other and exposed to water, nitric oxide was generated.

### Example II: Composite Article

A cellulose carrier was saturated with an aqueous solution containing sodium nitrite. Another cellulose carrier was saturated with an aqueous solution containing reduced glutathione and zinc chloride. The two layers were stacked on top of each other to form the composite article. The composite article was then exposed to water and generated nitric oxide by formation of S-nitrosoglutathione, which then rapidly decomposed to release nitric oxide (see FIG. 10). Alone, neither layer generated nitric oxide. Further, without the exposure to water, the layers together did not generate nitric oxide. However, when both layers were in contact with each other and exposed to water, nitric oxide was generated.

### Example III: Composite Article

An ethylene vinyl acetate carrier was saturated with sodium nitrite. Another ethylene vinyl acetate carrier was saturated with reduced glutathione. The two layers were stacked on top of each other to form the composite article. The composite article was then exposed to water and generated nitric oxide by formation of S-nitrosoglutathione, which then rapidly decomposed to release nitric oxide (see FIG. 11). Alone, neither layer generated nitric oxide. Further, without the exposure to water, the layers together did not generate nitric oxide. However, when both layers were in contact with each other and exposed to water, nitric oxide was generated.

It is to be understood that the appended claims are not limited to express and particular compounds, compositions, or methods described in the detailed description, which may vary between particular embodiments which fall within the scope of the appended claims. With respect to any Markush groups relied upon herein for describing particular features or aspects of various embodiments, different, special, and/or unexpected results may be obtained from each member of the respective Markush group independent from all other Markush members. Each member of a Markush group may be relied upon individually and or in combination and provides adequate support for specific embodiments within the scope of the appended claims.

Further, any ranges and subranges relied upon in describing various embodiments of the present invention independently and collectively fall within the scope of the appended claims, and are understood to describe and contemplate all ranges including whole and/or fractional values therein, even if such values are not expressly written herein. One of skill in the art readily recognizes that the enumerated ranges and subranges sufficiently describe and enable various embodiments of the present invention, and such ranges and subranges may be further delineated into relevant halves, thirds, quarters, fifths, and so on. As just one example, a range “of from 0.1 to 0.9” may be further delineated into a lower third, i.e., from 0.1 to 0.3, a middle third, i.e., from 0.4 to 0.6, and an upper third, i.e., from 0.7 to 0.9, which individually and collectively are within the scope of the appended claims, and may be relied upon individually and/or collectively and provide adequate support for specific embodiments within the scope of the appended claims. In addition, with respect to the language which defines or modifies a range, such as “at least,” “greater than,” “less than,” “no more than,” and the like, it is to be understood that such language includes subranges and/or an upper or lower limit. As another example, a range of “at least 10” inherently includes a subrange of from at least 10 to 35, a subrange of from at least 10 to 25, a subrange of from 25 to 35, and so on, and each subrange may be relied upon individually and/or collectively and provides adequate support for specific embodiments within the scope of the appended claims. Finally, an individual number within a disclosed range may be relied upon and provides adequate support for specific embodiments within the scope of the appended claims. For example, a range “of from 1 to 9” includes various individual integers, such as 3, as well as individual numbers including a decimal point (or fraction), such as 4.1, which may be relied upon and provide adequate support for specific embodiments within the scope of the appended claims.

The present invention has been described herein in an illustrative manner, and it is to be understood that the terminology which has been used is intended to be in the nature of words of description rather than of limitation. Many modifications and variations of the present invention are possible in light of the above teachings. The present invention may be practiced otherwise than as specifically described within the scope of the appended claims. The subject matter of all combinations of independent and dependent claims, both single and multiple dependent, is herein expressly contemplated.

What is claimed is:

1. A composite article comprising:
a source layer comprising a nitric oxide precursor; and
an activation layer overlying the source layer and comprising a thiol-containing compound;
wherein the nitric oxide precursor and the thiol-containing compound are capable of reacting in the presence of a solvent to form nitric oxide.

2. The composite article of claim 1, wherein the nitric oxide precursor and the thiol-containing compound are capable of reacting in the presence of the solvent to form a nitrosothiol, and wherein the nitrosothiol is capable of decomposing to form the nitric oxide.

3. The composite article of claim 1 or 2, wherein the nitric oxide precursor comprises a nitrite.

4. The composite article of claim 3, wherein the nitrite is selected from the group of sodium nitrite, sodium nitrite, calcium nitrite, potassium nitrite, tetrabutylammonium nitrite, dicyclohexylammonium nitrite, butylnitrite, isobutylnitrite, t-butylnitrite, amylnitrite, pentylnittrite, nitrite salts, ion paired nitrite, silver nitrite, zinc nitrite, iron nitrite, copper nitrite, transition metal-nitrite compounds, and combinations thereof.

5. The composite article of any one of claims 1-4, wherein the thiol-containing compound comprises a cysteine or derivative thereof, a thiol-derivatized polymer or filler, or a combination thereof.

6. The composite article of any one of claims 1-5, wherein the cysteine or derivative thereof is selected from the group of cysteine, glutathione, acetyl cysteine, penicillamine, acetylpenicillamine, S-nitroso-n-acetylpenicillamine, bucillamine, and combinations thereof.

7. The composite article of any one of claims 1-6 further comprising a catalyst.

8. The composite article of claim 7, wherein the catalyst comprises a transition metal, a non-metal, or a combination thereof.

9. The composite article of claim 8, wherein the catalyst comprises a transition metal selected from the group of copper (Cu), zinc (Zn), silver (Ag), gold (Au), lead (Pb), platinum (Pt), iron (Fe), magnesium (Mg), manganese (Mn), cobalt (Co), nickel (Ni), and combinations thereof.

10. The composite article of claim 9, wherein the catalyst is zinc chloride.

11. The composite article of claim 8, wherein the catalyst comprises a non-metal selected from the group of selenium (Se), tellurium, (Te), organometal compound, and combinations thereof.

12. The composite article of claim 11, wherein the catalyst is selected from the group of selenium, an organoselenium, and a combination thereof.

13. The composite article of any one of claims 7-12, wherein the activation layer further comprises the catalyst.

14. The composite article of any one of claims 7-12 further comprising a catalytic layer, wherein the catalytic layer comprises the catalyst.

15. The composite article of claim 14, wherein the catalytic layer is:
(a) disposed between the source layer and the activation layer;
(b) disposed on the source layer and optionally spaced from the activation layer;
(c) disposed on the activation layer and optionally spaced from the source layer; or
(d) combinations thereof.

16. The composite article of any one of claims 1-15, wherein at least one of the source layer or the activation layer comprises a carrier material formed from a cellulose, a polyvinyl chloride, a polyurethane, a carbosil, a polydimethylsiloxane, an acrylic polymer, a polyester, a poly(lactic acid), a poly(lactic-co-glycolic acid), poly(vinyl acetate), ethylene vinyl acetate, tecothane, pellethane, a hydrogel, a polytetrafluoroethylene, a copolymer thereof, or combinations thereof.

17. The composite article of claim 16, wherein the carrier material is formed from a cellulose.

18. The composite article of claim 16, wherein the carrier material is formed from a hydrogel selected from the group of a polymacron, a polyacrylamide, a collagen, an agarose, a hyaluronic acid, a poly(organophosphazenes), a chitosan, a poly(ethylene glycol), poly(vinyl alcohol), and combinations thereof.

19. The composite article of any one of claims 16-18, wherein the carrier material comprises:
a support comprising polytetrafluoroethylene in the form of a mesh or a fiber; and
a material disposed within the support and formed from a cellulose, a polyvinyl chloride, a polyurethane, a carbosil, a polydimethylsiloxane, an acrylic polymer, a polyester, a poly(lactic acid), a poly(lactic-co-glycolic acid), poly(vinyl acetate), ethylene vinyl acetate, tecothane, pellethane, a hydrogel, another polytetrafluoroethylene, a copolymer thereof, or combinations thereof.

20. The composite article of any one of claim 1-19, wherein at least one of the source layer or the activation layer has a permeability in an amount of at least 0.001 g/(m·s·Pa) in accordance with ASTM E2945-14(2021) for permitting movement of at least one of the nitric oxide precursor or the solvent to or through the activation layer.

21. The composite article of claim 20 further comprising a metering layer capable of modulating the movement of at least one of the nitric oxide precursor or the solvent to or through the activation layer.

22. The composite article of claim 21, wherein the metering layer is:
(a) disposed between the source layer and the activation layer;
(b) disposed on the source layer and optionally spaced from the activation layer;
(c) disposed on the activation layer and optionally spaced from the source layer; or
(d) combinations thereof.

23. The composite article of claim 22, wherein the metering layer is disposed on the activation layer and spaced from the source layer, and wherein the metering layer only partially overlies the activation layer such that a portion of the activation layer is free of the metering layer.

24. The composite article of any one of claims 21-23, wherein the metering layer has a permeability different than the permeability of at least one of the source layer or the activation layer.

25. The composite article of claim 24, wherein the metering layer has a permeability in an amount of at least 0.001 g/(m·s·Pa) in accordance with ASTM E2945-14(2021), but less than the permeability of at least one of the source layer or the activation layer.

26. The composite article of any one of claims 1-25, wherein the source layer and the activation layer are in direct contact.

27. The composite article of any one of claims 1-26, wherein the solvent comprises water.

28. The composite article of any one of claims 1-27 further comprising an additive selective from the group of transition metals, ascorbic acid, pH controlling layers, buffering components.

29. The composite article of claim 28, wherein at least one of the source layer or the activation layer comprises one or more of the additives.

30. The composite article of any one of claims 1-29, wherein the nitric oxide is capable of being formed:
(a) at a temperature of from 0° C. to 100° C.;
(b) in the presence or absence of visible light; or
(c) a combination thereof.

31. A food packaging article capable of forming nitric oxide to preserve foodstuff, comprising:
a source layer comprising a nitric oxide precursor;
an activation layer overlying the source layer and comprising a thiol-containing compound; and
a contact layer overlying the activation layer;
wherein the nitric oxide precursor and the thiol-containing compound are capable of reacting in the presence of a solvent to form nitric oxide.

32. The food packaging article of claim 31, wherein the contact layer has a permeability in an amount of at least 0.001 g/(m·s·Pa) in accordance with ASTM E2945-14(2021) for permitting movement of nitric oxide therethrough.

33. The food packaging article of claim 32, wherein the contact layer is substantially impermeable to ions.

34. The food packaging article of any one of claims 31-33 further comprising a barrier layer, wherein the source layer is disposed on the barrier layer and the barrier layer is spaced from the activation layer.

35. The food packaging article of claim 34, wherein the barrier layer has a permeability in an amount of less than 0.01 g/(m·s·Pa) in accordance with ASTM E2945-14(2021) to prevent movement of nitric oxide therethrough.

36. The food packaging article of any one of claims 31-35, wherein the food packaging article is in the form of a rigid container, wrapper, bag, bottle, or tube.

37. The food packaging article of any one of claims 31-36, wherein poultry contained within the food packaging article exhibits a decrease in spoilage after 96 hours at 23° C. as compared to poultry contained with a container free of at least one of the nitric oxide precursor or the thiol-containing compound.

38. A vehicle headliner of a vehicle capable of forming nitric oxide to sanitize the vehicle, comprising:
a source layer comprising a nitric oxide precursor;
an activation layer overlying the source layer and comprising a thiol-containing compound; and
a fabric layer overlying the activation layer;
wherein the nitric oxide precursor and the thiol-containing compound are capable of reacting in the presence of a solvent to form nitric oxide.

39. A composition comprising:
a source portion comprising a nitric oxide precursor; and
an activation portion comprising a thiol-containing compound;
wherein the nitric oxide precursor and the thiol-containing compound are capable of reacting in the presence of a solvent to form nitric oxide.

40. A washing machine component capable of forming nitric oxide to resist growth of mold or mildew, comprising:
a source portion comprising a nitric oxide precursor; and
an activation portion comprising a thiol-containing compound;
wherein the nitric oxide precursor and the thiol-containing compound are capable of reacting in the presence of a solvent to form nitric oxide.

41. The washing machine component of claim 40, wherein the washing machine component is a gasket or a liner.

42. A sealant composition capable of forming nitric oxide to resist growth of mold or mildew, comprising:
a source portion comprising a nitric oxide precursor;
an activation portion comprising a thiol-containing compound; and
a sealant material;
wherein the nitric oxide precursor and the thiol-containing compound are capable of reacting in the presence of a solvent to form nitric oxide.

43. The sealant composition of claim 42, wherein the sealant material is selected from the group of a silicone, an epoxy, a polyurethane, a polysulfide, a latex, and combinations thereof.
